(19) United States
(12) Patent Application Publication
(10) Pub. No.: US 2021/0304882 A1
(43) Pub. Date: Sep. 30, 2021

(54) SYSTEM AND METHOD FOR OPTIMIZING THE PREPARATION OF PATIENTS FOR THEIR SURGERIES, ENDOSCOPIC AND INVESTIGATIVE PROCEDURES

(71) Applicant: DCB DIAMOND CRÈME BRÛLÉE DESIGN INC., Perth (CA)

(72) Inventor: Alain GAUTHIER, Perth (CA)

(21) Appl. No.: 17/219,772

(22) Filed: Mar. 31, 2021

(30) Foreign Application Priority Data
Mar. 31, 2020 (CA) 3077554

(51) Int. Cl. G16H 40/20; G16H 20/10; G16H 40/63; G16H 40/67; G16H 20/40

(52) U.S. Cl. CPC G16H 40/20; G16H 20/10; G16H 20/40; G16H 40/67; G16H 40/63

(57) ABSTRACT

A computer-implemented system and method for optimizing clinical process flow and operational efficiencies regarding the preparation of patients for their surgeries, endoscopic and investigative procedures, and for generating pre-operation instructions specific to each patient in various settings including hospitals, pre-operative clinics, physician's offices, nurse practitioner's offices, physician and health care providers, and other environments where patient and care provider encounters take place.

### CROSS-REFERENCE TO RELATED APPLICATIONS

This application claims priority to CA Patent Application No. 3,077,554, filed on Mar. 31, 2020, the contents of which are incorporated herein by reference in their entirety.

### BACKGROUND OF THE INVENTION

### Field of the Invention

The present invention in general to the field of healthcare systems and, more particularly, to a computer-implemented system and method for optimizing clinical process flow and operational efficiencies for the preparation of patients for their surgeries, endoscopic and investigative procedures. The present invention further relates to a computer-implemented system and method for generating pre-operational instructions specific to each patient in various settings including hospitals, pre-operative clinics, physician's offices, nurse practitioner's offices, physician and health care provider, and any other environments where patient and care provider encounters will take place.

### Description of Related Art

Success of medical procedures, such as surgeries, endoscopic and investigative procedures not only rely on professional skills of the medical professionals but also on patients, i.e., how well they were prepared for such operations. For optimal outcomes, the patients would have typically been given instructions before their surgeries, endoscopic and investigative procedures in the form of general verbal and/or printed instructions. For simplicity to the medical professionals, these instructions are typically broad and non-specific to the individual patients.

If patients undergo these procedures while not being optimally prepared, their outcomes may be suboptimal. In certain situations, such procedures may often need to be cancelled and postponed, causing significant physical and mental stresses to these patients, delays in diagnosis and treatments as well as increased cost to the health care system.

Currently, the instructions typically provided by the medical professionals, health care providers and surgeons are often confusing for many of their patients as these instructions are very generic and not specific or applicable to specific patients as shown in FIG. 1. This is particularly true when it comes to instructions related to medications, natural remedies, vitamins and supplements.

When patients come to the operating room, endoscopy clinic or imaging departments, the patients need to be properly prepared prior to these procedures. This often means that the patients should have stopped certain medications before these procedures and should have continued taking other medications up to and including on the day of their procedures. For example, certain types of medications may increase bleeding such as anti-coagulants and anti-platelets and as such would need to be stopped before these procedures, otherwise they may cause significant complications such as difficult to control bleeding and increase the need for blood transfusions. On the other hand, medications for controlling blood pressure often should be taken on the day of these procedures as to decrease events such as intra-operative cardiac adverse events including myocardial infarcts. Other medications dosage such as insulin need to be adjusted according to the individual, their situation and the other medications they take for their diabetes. If the management of these medication prior to these procedures is not properly done this may lead to increased risks of adverse effects associated to either to low or elevated blood glucose.

As an example, FIG. 1 illustrates an existing exemplary set of patient preparation instructions for bowel surgery—right colectomy, which shows the reasons why typical general instructions provided by surgeons are often very confusing to patients due to the fact that these typical exemplary instructions are too general, and thus, lack specifics for any particular patient. It can also be seen that these instructions are often long, written in smaller fonts, hard to read for patients with minimum or limited knowledge regarding medical terms, and contain various irrelevant information that may not be applicable to a particular patient. For example, these instructions often include information specific to patients with diabetic medications or/and with medications affecting the coagulation cascade or platelets with medications such as Coumadin, Pradaxa, and Plavix. When patients do not have any of these conditions, the printed information is irrelevant for them and, thus, may only add to their possible confusion. Due to the fact that such typical instructions contain various irrelevant information, many of the patients may not even take their time to read through these instructions carefully.

In addition, each medication often has multiple names, including generic names and brand names. Accordingly, many patients are often unable to recognize the names that are equivalent to the ones that they are taking. Furthermore, many of the patients would not even realize that some or all of the instructions were applicable to their situation(s). For example, in FIG. 1, Coumadin® (brand name) is listed as a medication with instructions but may not be recognized by the patients if they only know the generic name of the medication, such as Warfarin®, or vice versa. Another example from FIG. 1 is Plavix® (brand name) which is also known as Clopidogrel® (generic name). Accordingly, relying on the patient's knowledge regarding medication often result in ineffective patient's preparation for the surgeries, endoscopic and investigative procedures.

In addition, FIG. 1 also shows the lack of specific instructions for most medications including insulin which need to be properly taken for optimal health outcomes. For example, where PRADAXA/XARELTO® are listed in FIG. 1 such as “family of pills”, such statement cannot be more confusing for patients. In this situation, a medical professional would understand that “family of pills” would include other medications such as Eliquis® (brand name) or its generic version named Apixaban®; however, this would not be easily understood by the patients with limited/minimum medicinal knowledge. Accordingly, this type of printed instructions makes it almost impossible for all of the patients to know and understand what they should be doing for the preparation if they take this medication.

Furthermore, medical professionals and health care workers often provide to their patients broad instructions to keep things simpler to put on a written handout. For example, and going back to FIG. 1, Pradaxa®, Xarelto® and well as Eliquis® should not be stopped 7 days before these procedures. These medications should be stopped 2, 3 or 4 days before the procedure according to which medication is involved but also depending on the renal function of the patient. In such situation patients with decreased renal function need to stop these specific medications earlier then patients with normal renal function with respect to the date of their procedure. Such case-by-case modifications of instructions are difficult to put down on pre-printed, non-patient specific and non-medication specific instructions.

Most physicians including surgeons are experts in their specific field. These physicians have to focus on their tasks and cannot know everything about every fields of medicine. For example, it would be extremely challenging for orthopedic surgeons who need to know about new surgical techniques in their field to also be experts and up to date with every new medication that may be used by patients giving the large of medical conditions patients may have. Such physicians need the help of other physicians to properly prepare their patients for their procedures. Computer-implemented systems, such as the current invention, frequently facilitate this type of task.

In addition, medical professionals like surgeons and physicians are often not be able to take appropriate and necessary time to prepare and update, for example, even printed instructions for patients' preparation before surgery or operation on a regular basis and/or as needed. Accordingly, this may cause some inadequacies in providing instructions to the patients, resulting in inefficiency for the patients' preparations.

Various attempts were previously made to address various issues that are identified herein. For example, U.S. Pat. No. 8,532,807 to Metzger discloses a pre-operative planning and manufacturing method for orthopedic surgery. US Patent Application Publication No. 2005/0273359 to Young discloses a health care screening system that obtains patient health history and analyzes data to determine recommended preoperative medical testing. However, as it can be seen, Metzger or Young does not provide any considerations regarding pre-operation preparation related to the medication(s) for the patients who are schedule for operations.

For another example, U.S. Pat. Nos. 9,129,054 and 10,166,019 issued to Nawana et al. disclose a systems and methods for surgical and interventional planning, support, post-operative follow-up, and, functional recovery tracking; however, Nawana et al. lack detailed considerations and an enabling disclosure of how any of the issues identified above can be mitigated and addressed.

Accordingly, there has been a long-felt need(s) for a system and/or method for addressing one or more of the presently identified issues.

### BRIEF SUMMARY OF THE INVENTION

Accordingly, an object of the present invention is to give individualized instructions to patients that will be specific to their own medications, natural remedies, vitamins and supplements in order to 1) maximize the outcomes of scheduled surgeries, endoscopic and investigative procedures, 2) prepare and optimize patients for their anesthesia and/or sedation and 3) to optimize the effects of any other treatment(s) that the patients may be receiving.

Another object of the present invention is to provide clear instructions for continuing and if applicable when to stop these medications, natural remedies, vitamins and supplements should be stopped with respect to the date of the procedures.

Yet another object of the present invention is to provide instructions for the patients whether to take their medications, natural remedies, vitamins and supplements if appropriate the evening before and on the morning of the procedure. In addition, the instructions further include other type of instructions, including but not limited to fasting instructions. Other example of such additional instructions may include instructions for the purchase of medically, travel arrangements and other precautions needed before or after their procedures.

According to one aspect of the present invention, it provides a system and method to provide and suggest to the used standard medically agreed upon instructions, including if and when they should take or stop their medications, to patients in preparations for surgeries, endoscopic and investigative procedures.

According to yet further aspect of the present invention, it provides a system and method to generate alternate instructions and will provide alternate option(s) to the user for each medications, natural remedy, vitamin and supplement which will allow easy modification on case-by-case when needed.

According to another aspect of the present invention, it provides medical professionals an improved system(s), process(es) and method(s) for updating the list of medications and instructions therefor to the patients in preparations for surgeries, endoscopic and investigative procedures.

According to yet another aspect of the present invention, it provides a simple step-by-step system and method for users to create a patient profile and be able to return to created profiles as needed.

According to further aspect of the present invention, it provides a system and method for a system and method for storing and managing documented records of the information given by the user to patients.

According to yet further aspect of the present invention, it provides a system and method to allow the user to specify and to enter information and data regarding the procedure, type and date for generating instructions to the patients in preparation for surgeries, endoscopic and investigative procedures.

According to yet further aspect of the present invention, it provides a system and method to allow the user to easily make selection of only the medications, natural remedies, vitamins and supplements that patients are specifically taking to generate customized and very specific instructions to the patient, without making any reference to other medications or items they are not taking.

According to yet further aspect of the present invention, it provides a system and method that provides both brand and generic names of medications, allowing the user or patients to enter their medication information easily to minimize data entry errors. By providing both type of names, it allows the patients or user with limited knowledge regarding medications to recognize and enter medication information easily and more accurately.

According to yet further aspect of the invention, it provides a system and method to generate final instructions to each patient that will be specific to their own situation as well as to the specific medications, natural remedies, vitamins and supplements he/she takes. The system and method of the present invention provide instructions to patients if and when they should reduce and/or stop their medications, natural remedies, vitamins and supplements ahead of their surgeries, endoscopic and investigative procedures. When appropriate, the system and method of the invention provide them instructions whether to take their medications, natural remedies, vitamins and supplements on the day of their surgeries, endoscopic and investigative procedures with specific dosages.

According to further aspect of the present invention, it provides a system and method that further generates additional instructions the patients, their care providers and/or other medical professionals, which may include but will not be limited to fasting instructions to the patients; instructions as to when patients should come to the place where their procedure will be done; instructions related to their level of activity before and after their procedures; instructions regarding appointment to care providers before the surgeries, endoscopic and investigative procedures; instructions to anaesthetists; and instructions to pre-op nurses.

According to another aspect of the current invention it will allow for the distribution of instructions via different means which may include but will not be limited to printed format, electronic messages and texts, schedule/calendar reminders and other types of reminders. Those other types of reminders may include, but not limited to, sending notifications, pop-ups and reminders to a mobile device(s) (including tablets and other personal computing devices), third-party computing or wearable devices that a user or the patient(s) may use, including, but not limited to mobile phones, smart watches, fitness tracker, or future wearable used for health monitoring, etc.

According to further aspect of the present invention, it provides a system and method that allow for the communication of these instructions to the patients themselves, to other care providers, including pharmacists, as to ensure compliance by the patients. This may include instructions for the development, management, and adjustment of compliance packaging of medications (aka blister packs or bubble packs). It further provides a system and method for allowing pharmacies to help patients plan medication transitions for preparation of their medical procedures, and further helping senior guide medication timing.

According to yet further aspect of the present invention, it provides a system, comprising a microprocessor that receives one or more names of medications prescribed to a patient; and, at least one data storage that is in communication with the microprocessor. The data storage stores and manages one or more medication rules. Each of said medication rules is for a particular medication, comprises one or more names of the particular medication and one or more patient preparation procedures. The microprocessor accesses the data storage and retrieves one or more of the medications rules that matches with one or more of the names of mediations to generate preparation instructions to the patient.

The microprocessor further receives one or more names of surgery or procedure scheduled for the patient. The data storage further stores and manages one or more surgery rules. Each of the surgery rules is for a particular surgery or medical procedure, comprise one or more names of the particular surgery or medical procedure and one or more patient preparation procedures therefor. The microprocessor accesses the data storage and retrieves one or more of the surgery rules that matches with the name(s) of surgery or operation for the preparation instructions to the patient.

The microprocessor further receives a scheduled date or scheduled dates of surgery or procedure scheduled for said patient for said preparation instructions to said patient.

The data storage is a local database, an external database, or a remote database server.

The system is a computing device, a web server, or an application server. The system is in communication with a remote user interface terminal for a user to enter the names of medications, and/or to enter said the names of surgeries or procedures scheduled for the patient.

One or more names of the particular medication include one or more brand names and/or one or more generic names of the particular medication. This would facilitate and help patients to directly contact a medical professional for clarifying any concerns the patient may have (for example, a chat or help feature within the system by providing links to a medical professional) and would further prevent the patients from frequently and directly reaching or contacting their doctor for clarifications.

### BRIEF DESCRIPTION OF THE SEVERAL VIEWS OF THE DRAWING

To provide a more complete understanding of the present disclosure and features and advantages thereof, reference is made to the following description, taken in conjunction with the accompanying figures, wherein like reference numeral represent like parts, in which:

FIG. 1 shows an existing exemplary patient preparation instruction sheet for bowel surgery—right colectomy;

FIG. 2 shows a block diagram of a networked system in accordance with the present invention;

FIG. 3 shows a block diagram of a computing device in accordance with the present invention;

FIG. 4 shows exemplary data structures for the system and method in accordance with the present invention;

FIG. 5 shows an exemplary process flow diagram in accordance with the present invention;

FIG. 6 shows an exemplary illustration of a user interface for logging in as a user with his/hers email address and password or creating a new user account. This figure also shows the process a user would use if he/she forgot their password.

FIG. 7 shows an exemplary illustration of a user interface for entering the user name, e-mail address, password, confirming the password, street address including town province and postal code, and phone number.

FIG. 8 shows an exemplary illustration of a user interface for entering the affiliated hospital name, address, city, province/state, country, postal code, phone number and logo.

FIG. 9 shows an exemplary illustration of a user interface for adding preparation documents.

FIG. 10 shows an exemplary illustration of a user interface for the type of subscription.

FIG. 11 shows an exemplary illustration of a user interface for creating/entering new patient's profile or returning to one of the existing profiles;

FIG. 12 shows an exemplary illustration of a user interface for entering a patient's information including but not limited to their name, date of birth, health card/insurance information, phone number, mobile phone number, e-mail address, name of family doctor, and pharmacy name and fax number;

FIG. 13 shows an exemplary illustration of a user interface for entering types of surgery/operation, planned date of the surgery/operation, renal status of the patient, etc.;

FIG. 14 shows an exemplary illustration of a user interface for entering/selecting the medications, natural remedies, vitamins and supplements taken by the patient;

FIG. 15 shows an exemplary illustration of a user interface for selecting the suggested recommendation for the management of a medication as well as alternative options to be selected on a case-by-case; and

FIG. 16 shows an exemplary illustration of a generated final instructions for the patient in accordance with the present invention.

### DETAILED DESCRIPTION OF THE INVENTION

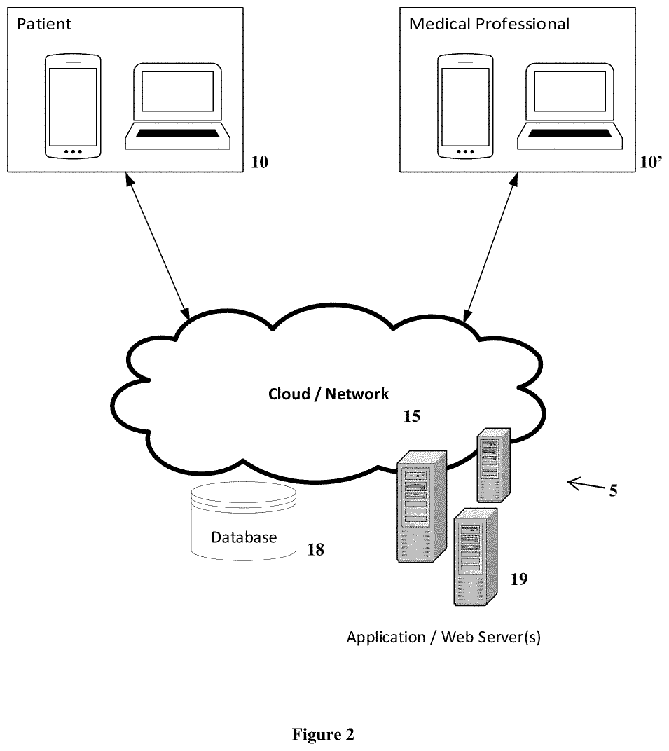

FIG. 2 shows a block diagram of a networked system 5 in accordance with the present invention. The networked system 5 include a plurality of computing devices 10, 10′ that interface with patients, users and medical professionals. The computing devices 10, 10′ may be desktop computers, laptops, tablets, cellphones, or other devices/apparatus. The computing devices 10, 10′ are in communication (through wired connection or wirelessly) with one or more web or application servers 19 and one or more remote database (database server, or data storage with a server) 18 in a network or cloud 15. The cloud 15 may be Amazon® Web Services (AWS), IBM® Cloud Compute or alike that provide cloud-based computing environment.

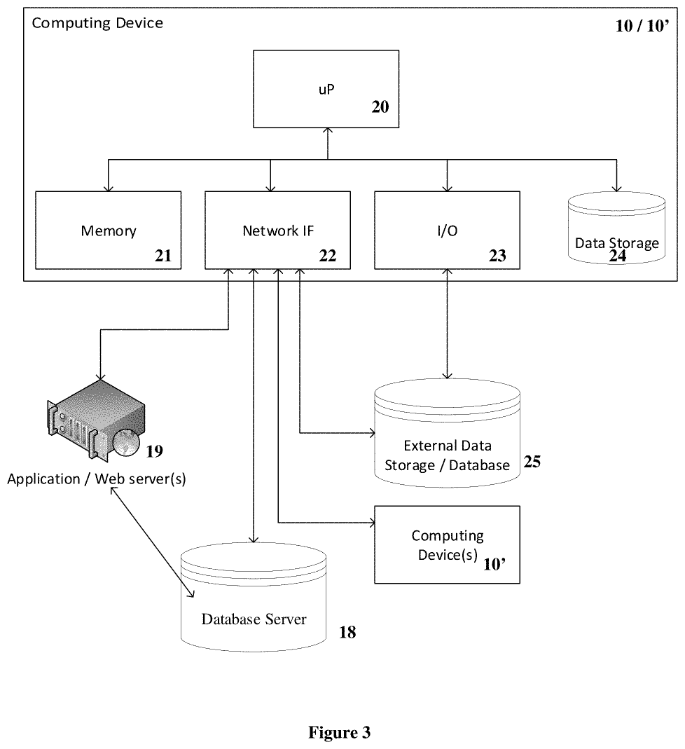

Referring to FIG. 3, the computing device 10 includes a processor/microprocessor 20, which is in communication with one or more memory(ies) 21 for storing temporary data and/or executable codes, including, but not limited to random access memory (RAM); a network interface module 22 including but not limited to wired network or wireless network interface; input/output module 23 for interfacing with various input/output devices such as USB devices, camera, video, monitor, printer, additional network interface module, additional external data storage device, etc.; and, data storage (or local data storage) 24 for storing data and/or executable code, including but not limited to non-volatile memory, flash memory, solid-state drives, etc. The microcontroller 20 of the computing device 10 may be in communication with an external data storage/database/database server 25 through the input/output module 23. The web/application server 19 would comprise a similar basic hardware structure to the computing device 10 or derivative therefrom. For example, the web/application server 19 may have multiple microprocessors, multiple memories, multiple network interface module, multiple 110 module, and multiple data storages which may be arranged to carry out large volume of computation requests from a number of client devices connected thereto.

Through the network interface module 22, the microprocessor 20 is in communication with the web/application server 19, remote data base 18 and/or other computing device(s) 10′.

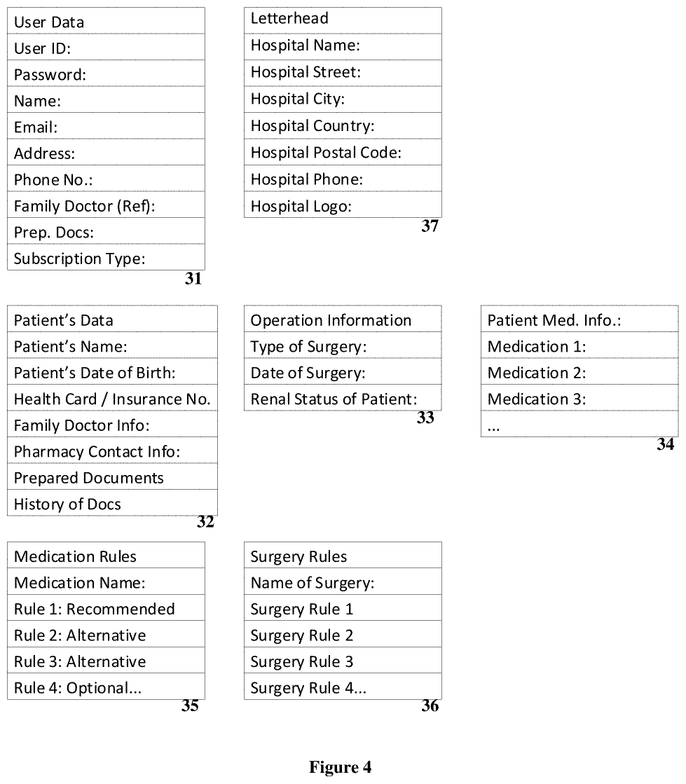

FIG. 4 illustrates an exemplary data structures for storing various data for the computing device 10 or web/application server(s) 19 to carry out the present invention. As a person of ordinary skilled in the pertinent art would understand that various other data structures may be used for carrying out the present invention without departing from the scope of the present invention. Such data structures include, not limited to user data 31 for storing data include, but not limited to user identification name (first name, middle name, last name, saltation, etc.), e-mail address, password (with confirmation), address, phone number(s), affiliated hospital (with address, city, province/state, country, postal code, phone number), logo, other patient preparation documents (templates) and subscription information and other details; patient's data 32 for storing data including, but not limited to patient's name, patient's date of birth, health card/insurance number or relevant information, family doctor and/or primary care provider information, pharmacy contact information (which may include the pharmacist's name, phone and fax numbers, e-mail address and other information), prepared document(s) (presently generated) and history of documents prepared by the system; surgery, endoscopic or investigative procedures information 33, including but not limited to type and the date of surgery, operation and/or procedure, and the renal status of the patient; patient's medication information 34, for storing and managing information regarding the patient's medication, natural remedies, vitamins and supplements; medication rules 35 for storing and managing one or more recommended rules, one or more alternative rules, and one or more optional rules for preparing a patient with a particular medication(s), natural remedy(ies), vitamin(s) and supplement(s) related to surgery/operation; and, surgery rules 36 for storing and managing one or more rules specific to a particular surgery or operation, such as fasting etc. Each record in the medication rules 35 may contain one or more names for a particular medication/remedy/vitamin/supplement, including one or more brand names and/or one or more generic names therefor; and one or more recommended rules, alternative rules and/or optional rules corresponding to the particular medication related to surgery/operation. Optionally, the data structures of the present invention, may, further comprises letterhead 37 for storing specific letterhead information for a specific hospital that uses the present invention for generating patient's instructions. The letterhead 37 includes hospital name, hospital address including street address, city, country, and postal code/zip code, hospital phone number(s) and logo/trademark or image that the hospital may use. The user data 31 may further include subscription type for storing and keeping track of use's subscription type for use of the present invention. Patient specific information would be stored and managed under the patient's data 32, operation information 33, and patient's medication information 34. The patient specific data, patient's data 32, operation information 33, and patient's medication information 34, may be stored and managed in the local data storage 24 of the patient's computing device 10; the local data storage 24 of the medical professional's computing device 10′; web/application server 19; or/and, remote database 18, depending on the implementation preferences or requirements.

As it can be seen, medical professional would enter, update and maintain various rules related to medication and patient preparation in medical rules 35 and surgery rules 36, as required and appropriate. Medical rules 35 and surgery rules 36 may be stored and managed in the local data storage 24 of the medical professional's computing device 10′; web/application server 19; or/and, remote database 18. Medical rules 35 and surgery rules 36 may also be stored in the local data storage 24 of the patient's computing device 10; however, such data may be updated arbitrarily or periodically through the cloud/network 15 from the medical professional's computing device 10′, web/application server 19, and/or remote database 18.

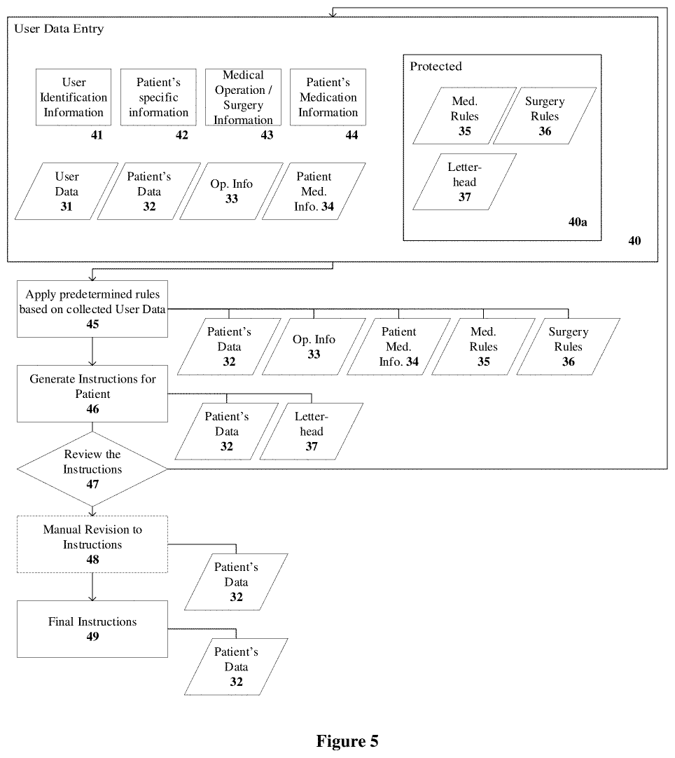

FIG. 5 shows an exemplary process flow diagram in accordance with the present invention. Initially, at step 40, the user, either or both a medical professional and patient enters user and patient related data. For example, the user may be a new or existing user of the system of the present invention, thus at sub-step 41, if new user, the user will enter user specific information, including, but not limited to user identification, password, personal name (first name, middle name, last name, saltation, etc.), email address, phone number(s), and/or family doctor information (name, address, and other contact information), which are stored/managed in the user data 31, or simply login/authenticated as an existing user based on the previously entered data stored/managed in the user data 31. In this context, the user may be a medical professional or a patient himself/herself.

At sub-step 42, if the existing patient, the system uploads the patient data including, but not limited to patient's name, patient's date of birth, health card/insurance number or relevant information, family doctor information, and pharmacy contact information, stored in the patient's data 32 such that the user may update the patient's specific data; or, if it is for a new patient, the user creates/enters patient's specific data in the patient's data 32.

At sub-step 43, if it is for an existing patient, the medical operation/surgery information is uploaded from the operation information 33 such that the user may update the medical operation/surgery information; or, if it is for a new patient or new operation/surgery, the user creates/enters new operation information in the operation information 33.

At sub-step 44, if it is for an existing patient, previously entered patient's medication information is uploaded from the patient's medication information 34 for the user to update; or, if it is for a new patient, the user creates/enters new medication information in the patient's medication information 34.

It is to be noted that order(s) of the sequence among sub-steps 42, 43 and 44 may not be essential to the working of the present invention, and, thus, the user may carry out in a random order, revisit or process multiple times at any one of sub-steps 42, 43 and 44. Optionally, a confirmation summary may be displayed for the user in order to correct any errors or omissions in the data entries.

Sub-step 40a is a protected step, where only a very specific and privileged user(s) may have access to enter/alter/delete data in the medication rules 35, surgery rules 36 and/or letterhead 37.

At step 45, based on the data entered at step 40, appropriate number of rules from the medication rules 35 and/or surgery rules 36 for constructing user specific instructions for the patient. Depending on, for example, various other patient's information entered in the patient's data 32, the operation information 33, and/or the patient's medication information 34, such as renal status of the patient, specific medication rule(s) for a particular medication may be chosen from the medication rules 35. Similarly, depending on, for example, various other patient's information entered in the patient's data 32, the operation information 33, and/or the patient's medication information 34, specific rules may be chosen from the surgery rules 36.

At step 46, specific instructions to the patient are generated based on the rules selected from medication rules 35 and surgery rules 36 based on the data entered in the patient's data 32, operation information 33, and patient's medication information 34. If appropriate, data from the letterhead 37 are used to customize its visual feature and information displayed in the generated instructions, including, but not limited to hospital name, address, phone number, and logo. Once the instruction is generated, such generated instructions may be stored as prepared documents and history of the documents in the patient's data 32.

Once the instructions are generated, the user (often a medical professional) reviews the instructions at step 47. If the instructions are not satisfactory, the process will be returned to step 40 for re-entering/revising the data. If the instructions generated is satisfactory, optionally, opportunity for the user (medical professional) to manually revise the instructions at step 48. Any manually revised instructions may further be stored and managed as prepared documents and history of the documents in the patient's data 32.

After the step 48, finalized instructions are generated at step 49 for the patient.

The present invention may be embodied in various different formats/structures/configurations. For example, according to a first exemplary embodiment of the present invention, the process steps shown in FIG. 5 may be carried out on the patient's computing device 10 (or medical professional's computing device 10′) alone. In this case, referring to FIGS. 2 and 3, the patient's data 32, operation information 33, patient's medication information 34 are stored and managed in the local data storage 24 of the computing device 10. The predetermined medication rules 35 and surgery rules 36 are also stored and managed in the local data storage 24 of the computing device 10. In this embodiment, it is optional to have user data 31, which may be stored and managed in the local data storage 24 of the computing device 10. The process steps as shown in FIG. 5 are processed and executed in the microprocessor 20 of the computing device 10.

According to a second exemplary embodiment of the present invention, referring to FIGS. 2 and 3, the process steps shown in FIG. 5 may be carried out partly on the patient's computer device 10 and/or the medical professional 10′ and partly on the web/application server 19. For example, all the data entry shown in the step 40 may be carried out on the patient's computer device 10 and/or the medical professional's computing device 10′, while application of the entered data to generate the instructions as shown in steps 45 and 46 may be carried out on the web/application server 19. The resulting instructions may be sent back via network to the patient's computer device 10 and/or the medical professional 10′ for it to carry out the remaining steps 47, 48 and 49. In this embodiment, the patient's data 32, operation information 33, and patient's medication information 34 may be stored and managed in either or both the patient's computer device 10 and/or the medical professional 10′ and the web/application server 19. Alternatively, the web/application server 19 may be in communication with the remote database server 18 for storing and managing the patient's data 32, operation information 33, and patient's medication information 34. The user data 31, in this embodiment, may be stored and managed at the web/application server 19 and/or remote database server 18. The medication rules 35 and surgery rules 36 would be stored and managed at either or both web/application server 19 and/or remote database server 18. The medical professional 10′ may only be able to create, edit and/or delete the medication rules 35 and surgery rules 36.

According to a third exemplary embodiment of the present invention, referring to FIGS. 2 and 3, the process steps shown in FIG. 5 may be carried out on the web/application server(s) 19, where the patient's computer device 10 and/or the medical professional 10′ merely work as a remote user interface terminal (or remote computing device) for displaying/receiving data entries. In this embodiment, the user data 31, the patient's data 32, operation information 33, and patient's medication information 34 as well as the medication rules 35 and surgery rules 36 would be stored and managed at either or both the web/application server 19 and/or remote database server 18, where the web/application server 19 is in communication with the remote database server 18. The medical professional 10′ may only be able to create, edit and/or delete the medication rules 35 and surgery rules 36.

According to a fourth exemplary embodiment of the present invention, referring to FIGS. 2 and 3, the process steps shown in FIG. 5 may be carried out on the patient's computer device 10 and/or the medical professional 10′, while the user data 31, the patient's data 32, operation information 33, and patient's medication information 34 as well as the medication rules 35 and surgery rules 36 may be stored and managed on either or both the web/application server 19 and/or remote database server 18. The medical professional 10′ may only be able to create, edit and/or delete the medication rules 35 and surgery rules 36.

As shown through various exemplary embodiments, there are many other variations of implementations or embodiments which are possible to carry out the present invention without departing the spirit of the present invention.

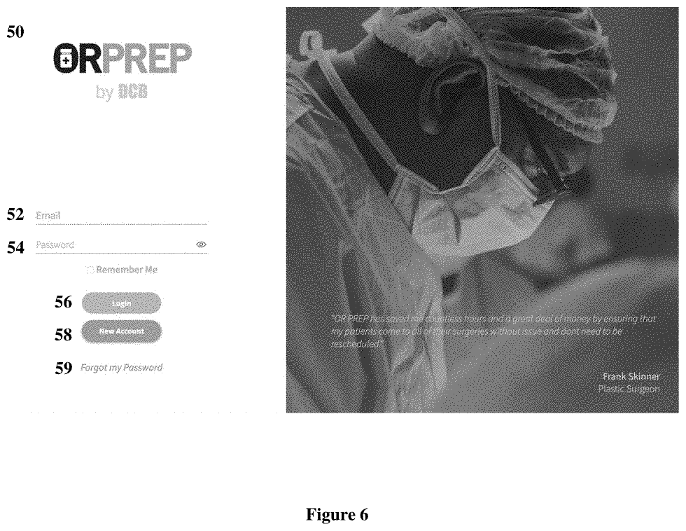

FIG. 6 shows an exemplary illustration of a user interface 50 for logging in a system in accordance with the present invention. User identification information is provided, including user ID 52, such as email address, and authentication information 54, such as password. An action button 56 or 58 causes the system to carry out the process sub-step 41 of the step 40. For example, action button 56 causes to carry out the process to compare entered user ID and authentication information against the user data 31 for granting an access; or, action button 58 causes to carry out the process of creating a new user account and entering data in user data 31. The interface 50 may further include an action button 59 that causes to carry out a process for a user to recover/reset password when/if he/she forgot their password.

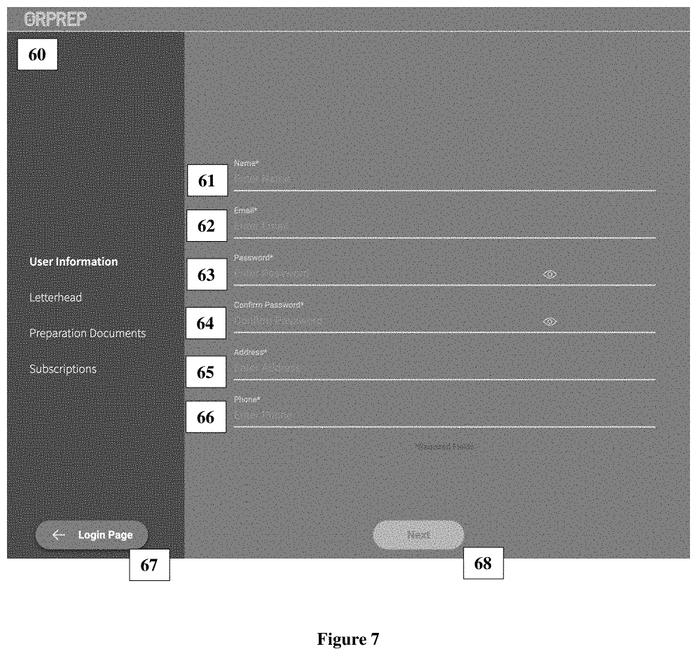

FIG. 7 shows an exemplary illustration of a user interface 60 for entering the user information including but not limited to a first field 61 for entering his/her name; a second field 62 for entering e-mail address; a third field 63 for entering a password; a fourth field 64 for confirming the password; a fifth field 65 for entering street address including town province and postal code; and, a sixth field 66 for entering phone number. An action button 67 causes to carry out a process to return to a previous user interface (or the user interface 50); and, another action button 68 causes to carry out a process of sub-step 41 of the step 40 for entering data in the user data 31, and proceed to a subsequent user interface.

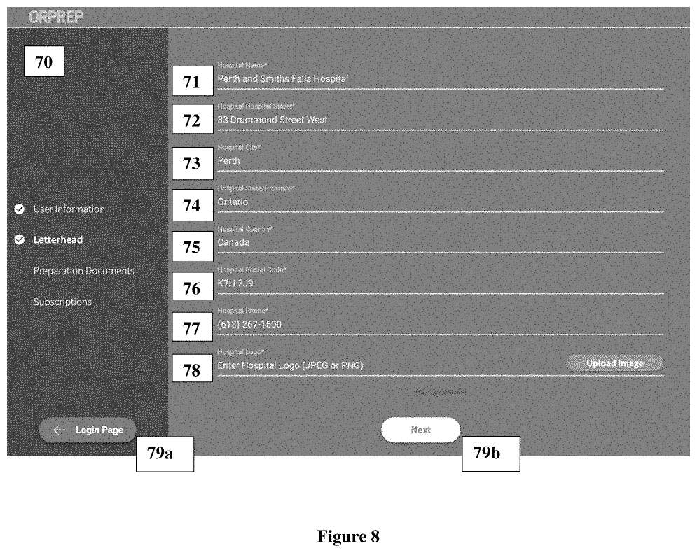

FIG. 8 shows an exemplary illustration of a user interface 70 for entering the information that will be part of the letterhead 37 including but not limited to a first field 71 for entering an affiliated hospital name; a second field 72 for entering hospital address; a third field 72 for hospital city 73; a forth field 74 for entering hospital province/state; a fifth field 75 for entering hospital country; a sixth field 76 for entering hospital postal code; a seventh field 77 for entering hospital phone number; and, a seventh field 78 for entering hospital logo. An action button 79a causes to carry out a process to return to a previous user interface (or the user interface 50 or 60); and, another action button 79b causes to carry out a process (or part thereof) of sub-step 40a of the step 40 for entering data in the letterhead data 37, and proceed to a subsequent user interface.

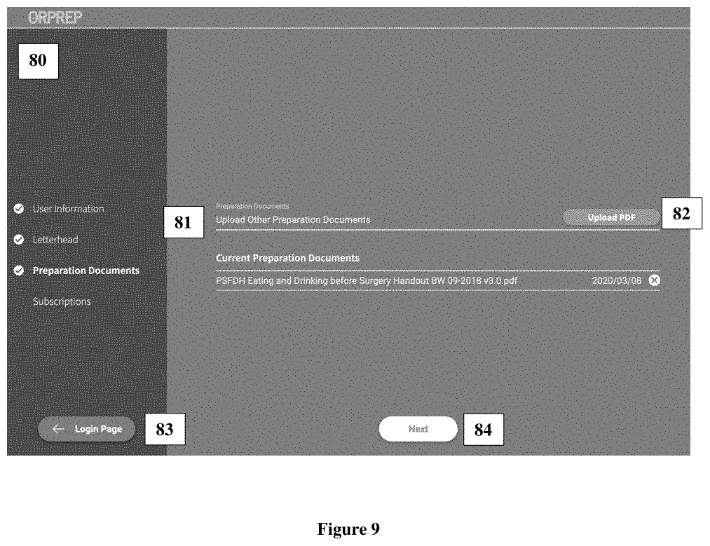

FIG. 9 shows an exemplary illustration of a user interface 80 for adding preparation documents (templates), including, but not limited to a field 81 for entering document information for uploading, and an action button 82 for causing to carry out a process of uploading the file specified to store in the user data 31. Another action button 83 causes to carry out a process to return to a previous user interface (or the user interface 50, 60 or 70); and, yet another action button 84 causes to carry out a process to proceed to a subsequent user interface.

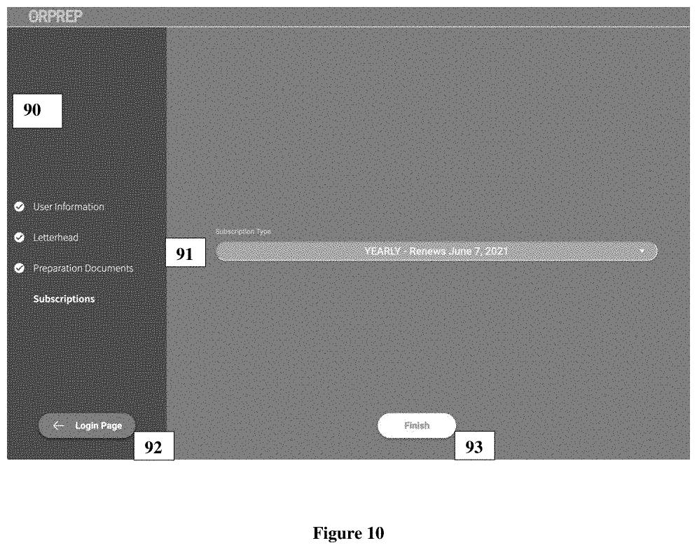

FIG. 10 shows an exemplary illustration of a user interface 90 for entering the type of subscription for the user, including, but not limited to, pull-down menu 91 with a list of selections for the user, causing to carry out a process of the sub-step 41 of the step 40 for updating subscription type information in the user data 31. An action button 92 causes to carry out a process to return to a previous user interface (or the user interface 50, 60, 70 or 80); and, yet another action button 93 causes to carry out a process to proceed to a subsequent user interface.

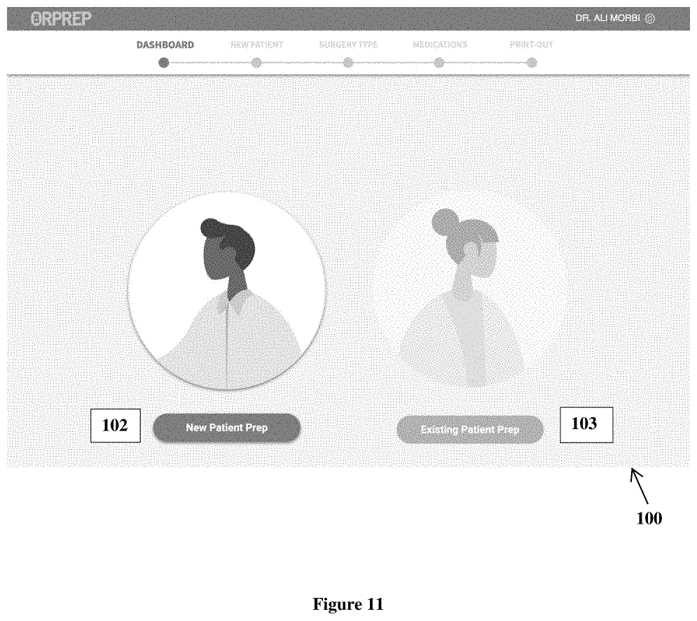

FIG. 11 shows an exemplary user interface 100 for selecting new patient or existing patient, including a first action button 102 for causing to start a process for creating a new patient; and, a second action button 103 for causing to start a process for retrieving an existing patient information.

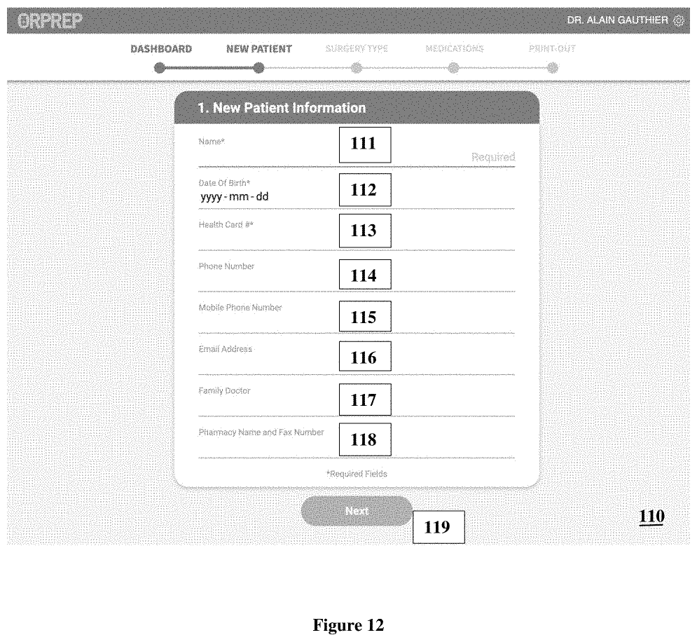

FIG. 12 shows an exemplary user interface 110 for entering data for a new patient in the patient's data 32 which includes, but not limited to a first field 111 for entering patient's name; a second field 112 for entering date of birth; a third field 113 for entering health card/insurance number; a fourth field for entering phone number 114; a fifth field 115 for entering mobile phone number; a sixth field 116 for entering e-mail address; a seventh field 117 for entering name of family doctor; and, an eighth field 118 for entering name and fax number of pharmacy. An action button 119 causes to carry out a process of the sub-step 42 to upload and save patient's data 32, and to proceed to a subsequent user interface. When the existing patient was chosen in the user interface 100, a similar user interface to the interface 110 would be displayed by uploading the existing patient data from the patient's data 32 and provide a user an opportunity to update any information thereof.

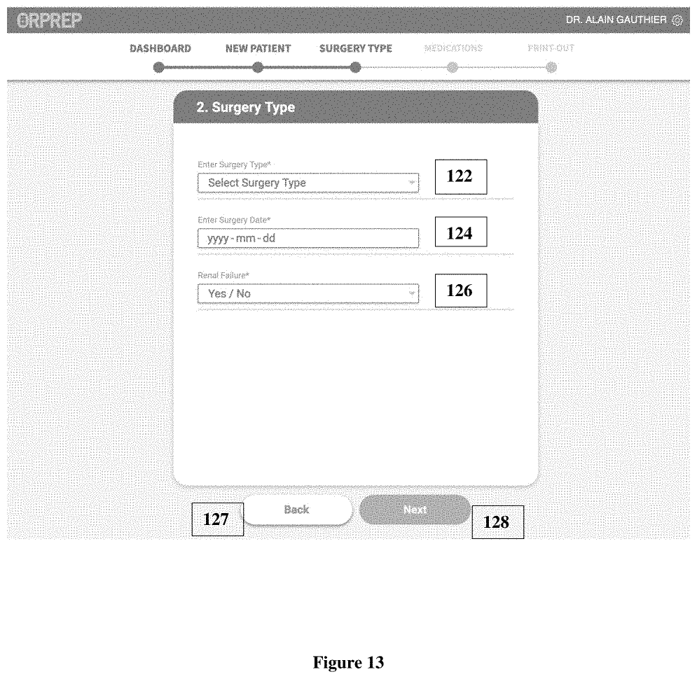

FIG. 13 shows an exemplary user interface 120 for entering data for the operation information 33, including but not limited to a first field (or pull-down menu) 112 for entering (or selecting) a type (which may be qualified as minor or major) of surgery the patient is scheduled to take; a second field 124 for entering scheduled date. In a preferred embodiment of the present invention, the interface 120 further provide a pull-down menu or field 126 for entering renal status of the patient. The renal status may be stored and managed in operation information 33, or in the patient's data 32. A first action button 127 causes to carry out a process to return to a previous user interface. A second action button 128 causes to carry out a process to upload and save data to operation information 33, and to proceed to a subsequent user interface.

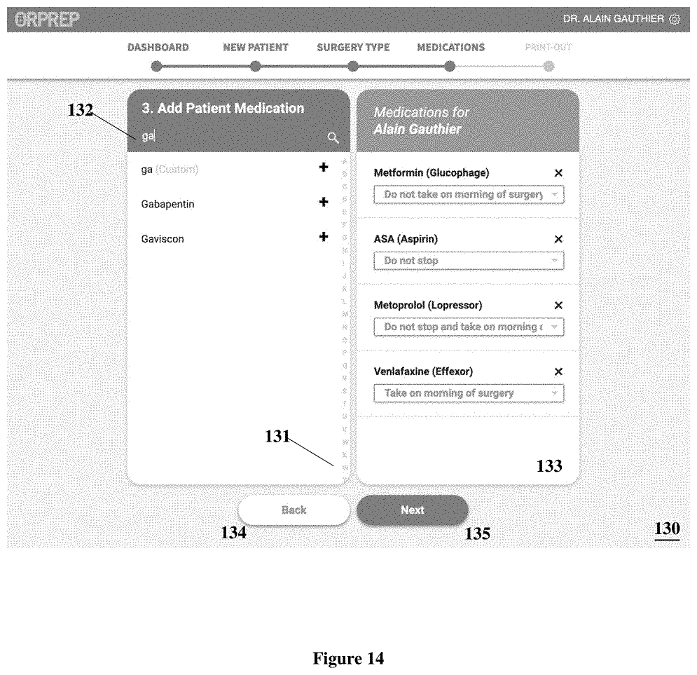

FIG. 14 shows an exemplary user interface 130 for entering data for the patient's medication information 34. This interface 130 may include a search window 132 which allows searches by typing the name of medication(s). The system finds the appropriate medication rule(s) from the medication rules 35 and display same on a sub-screen 133. A first action button 134 causes to carry out a process to return to a previous user interface. A second action button 135 causes to carry out a process to upload and save data to patient medication information 34, and to proceed to a subsequent user interface. Entering the patient's medication information 34 may also be automated by use of, for example, means (not shown) provided by the user interface 130, where the means would carry out optical character recognition capabilities to automatically capture and recognize the name(s) of the patient's medication(s) from a patient's medication report or list of prescription drugs that were generated by a pharmacist, physician, nurse, other health care provider or any person under their supervision. The report of the patient's medications, natural remedies, vitamins, supplements may be in a printed form (which may be scanned and converted into an electronic format for further processing), PDF format, or any other electronic format. This function would allow the user to enter the patient's list of medications, natural remedies, vitamins, supplements without having to use a keyboard in order to minimize any data entry error(s).

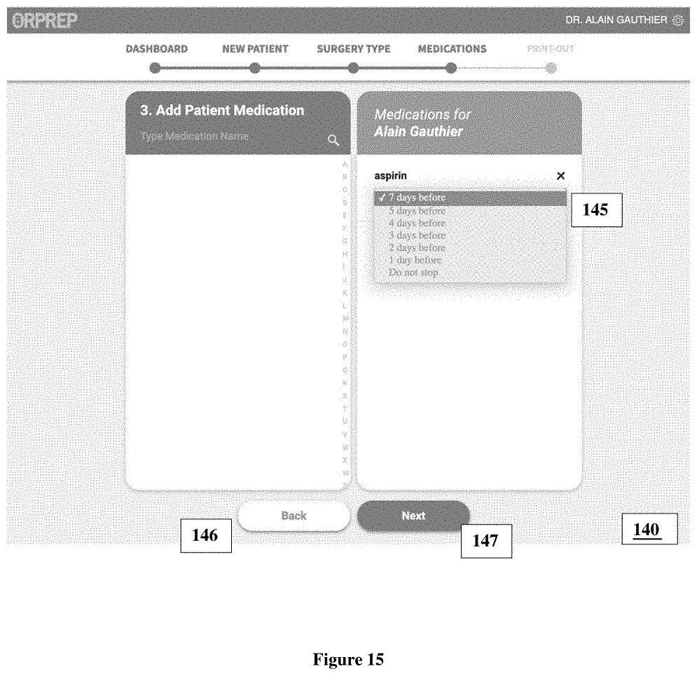

FIG. 15 shows an exemplary user interface 140 with the suggested recommendation for the management of a medication pre-selected as well as alternative options (in a drop-down menu) 145 to be selected on a case-by-case. A first action button 146 causes to carry out a process to return to a previous user interface. A second action button 147 causes to carry out a process to upload and save data to patient medication information 34, and to proceed to a subsequent user interface.

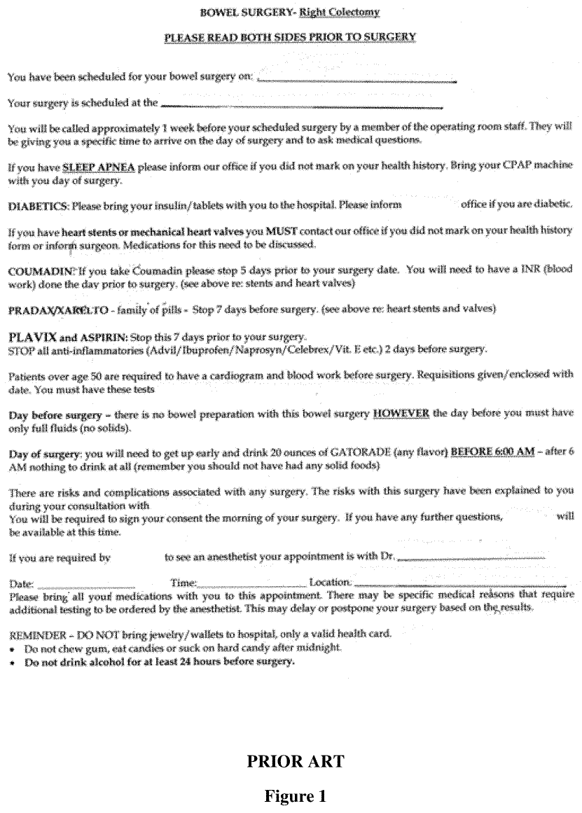

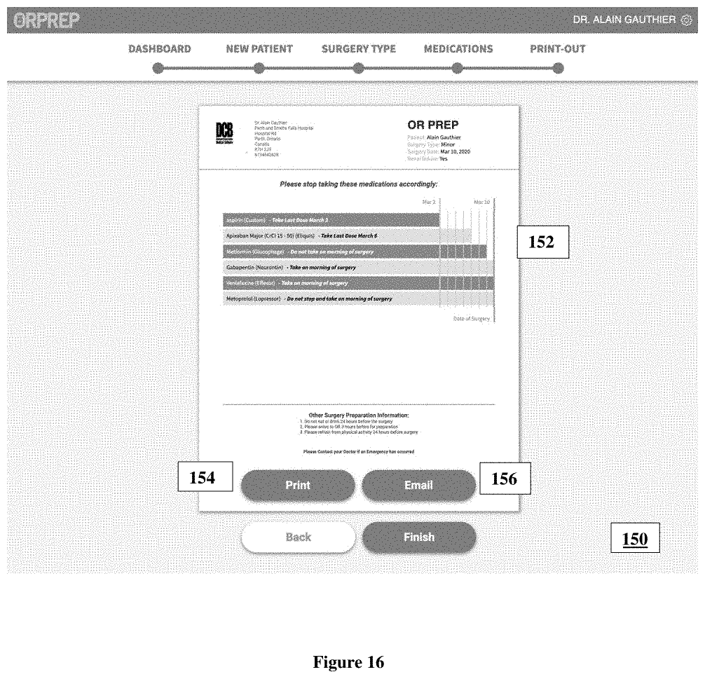

FIG. 16 shows an exemplary user interface 150 for displaying the resulting instructions based on entered data. The user interface 150 provides visualized and specific preparation instructions and schedule 152 for the patient which may be printed 154 or sent by e-mail 156 or communicated to the patient via other means (not shown). Unlike the prior art printed instructions shown in FIG. 1, the resulting visualized and specific preparation instructions and schedule 152 are specific to the name(s) of the medication(s), natural remedy(ies), vitamin(s) and/or supplement(s) that is/are taken by the patient based on the patent medication information 34, and specific to the preparation for his/her medical procedure. The visualized and specific preparation instructions and schedule 152 promotes maximizing the chance of success for the scheduled surgery/procedure, optimizing the patients for their anesthesia and/or sedation, and/or minimizing any impact on any other medical treatment(s) that the patients may be receiving by preventing unnecessary reduction/stoppage of the medications/natural remedies/vitamins/supplements such that optimizing the effects of those other medical treatment(s).

1. A system for generating preparation instructions specific for a patient, comprising:
a. a microprocessor that receives one or more names of medications prescribed to said patient; and,
b. at least one data storage that is in communication with the microprocessor, wherein said at least one data storage stores one or more medication rules, wherein each of said medication rules is for a particular medication and comprises one or more names for said particular medication and one or more patient preparation procedures for said particular medication,
wherein said microprocessor accesses said at least one data storage and retrieves one or more of said one or more medication rules that matches with said one or more names of mediations prescribed to said patient to generate said preparation instructions specific to said patient.

2. The system as recited in claim 1, wherein:
a. said microprocessor further receives one or more names of surgeries or procedures scheduled for said patient; and
b. said at least one data storage further stores one or more surgery rules, wherein each of said one or more surgery rules is for a particular surgery or medical procedure and comprises one or more names of said particular surgery or medical procedure and one or more patient preparation procedures for said particular surgery or medical procedure;
wherein said microprocessor accesses said at least one data storage and retrieves one or more of said one or more surgery rules that matches with said one or more names of surgeries or operations scheduled for said patient for said preparation instructions to said patient.

3. The system as recited in claim 2, wherein said microprocessor further receives a scheduled date or scheduled dates of a surgery or procedure scheduled for said patient for said preparation instructions to said patient.

4. The system as recited in claim 3, wherein said at least one data storage is a local database, an external database, or a remote database server.

5. The system as recited in claim 4, wherein said system is a computing device.

6. The system as recited in claim 4, wherein said system is a web server or an application server.

7. The system as recited in claim 6, wherein the system is in communication with a remote user interface terminal for a user to enter said one or more names of medications.

8. The system as recited in claim 7, wherein said remote user interface terminal is for said user to enter said one or more names of surgeries or procedures scheduled for said patient.

9. The system as recited in claim 1, wherein said one or more names of said particular medication include one or more brand names and/or one or more generic names of said particular medication.
